(19) United States
(12) Patent Application Publication
(10) Pub. No.: US 2007/0232972 A1
(43) Pub. Date: Oct. 4, 2007

(54) Agachaflex

(76) Inventor: Kelvin Martinez, Palm Coast, FL (US)

Correspondence Address: KELVIN MARTINEZ, 8 VILLAGE CIRCLE DR., PALM COAST, FL, 32164, US

(21) Appl. No.: 11/729,571

(22) Filed: Mar. 29, 2007

Related U.S. Application Data
(60) Provisional application No. 60/787,484, filed on Mar. 30, 2006.

(51) Int. Cl. A61F 5/00

(52) U.S. Cl. 602/16; 602/23; 602/27

(57) ABSTRACT

An apparatus which includes a strut system combined with an adjustable hinge system which substantially prevents or rehabilitates damage to the knee, especially due to frequent or prolonged periods of squatting or crouching. The strut system compresses while the weight of the body is absorbed by the combination of the strut and hinge system thus continuing to relieve the knee joint of excessive pressure during the entire squat or crouch. The effective life and maximum mobility of the knee is thus extended. Prolonged or even frequent crouching or squatting can be performed without the tiring out of the knee joint, discomfort, pain or damage to the knee. Subsequently, when the wearer stands up, and the knee is substantially extended, the hinge and strut system can aid in the lift of the body as the springs recoil while the strut provides additional lift support to the knees.

### CROSS REFERENCE TO RELATED APPLICATIONS

This application is based on provisional application Ser. No. 60/787,484, filed on Mar. 30, 2006.

### STATEMENT REGARDING FEDERALLY SPONSORED RESEARCH OR DEVELOPMENT

Not Applicable

### REFERENCES CITED

### U.S. Patent Documents

| | | |
|---|---|---|
| 7097627 | August 2006 | Enzerink et. al. |
| 6890314 | May 2005 | Seligman |
| 6878126 | April 2005 | Nelson et al. |
| 6821261 | November 2004 | Doty et al. |
| 6770045 | August 2004 | Naft et al. |
| 6666837 | December 2003 | Weihermuller |
| 6540709 | April 2003 | Smits |
| 6413232 | July 2002 | Townsend et al. |
| 6402711 | June 2002 | Nauert |
| 6383156 | May 2002 | Enzerink et al. |
| 6325773 | December 2001 | Opel |
| 5921946 | July 1999 | Tillinghast et al. |
| 5885235 | March 1999 | Opahle et al. |
| 5827208 | October 1998 | Mason et al. |
| 5817040 | October 1998 | Hess et al. |
| 5814000 | September 1998 | Kilbey |
| 5716336 | February 1998 | Hines et al. |
| 5672152 | September 1997 | Mason et al. |
| 5669873 | September 1997 | Towsley |
| 5658243 | August 1997 | Miller et al. |
| 5658241 | August 1997 | Deharde |
| 5653680 | August 1997 | Cruz |
| 5645524 | July 1997 | Doyle |
| 5632725 | May 1997 | Silver et al. |
| 5571078 | November 1996 | Malewicz |
| 5547464 | August 1996 | Luttrell et al. |
| 5460599 | October 1995 | Davis et al. |
| 5437619 | August 1995 | Malewicz et al. |
| 5409449 | April 1995 | Nebolon |
| 5360394 | November 1994 | Christensen |
| 5316547 | May 1994 | Gildersleeve |
| 5292303 | March 1994 | Bastyr et al. |
| 5244455 | September 1993 | Swicegood et al. |
| 5138911 | August 1992 | Lan |
| 5074290 | December 1991 | Harris et al. |
| 5052379 | October 1991 | Airy et al. |
| 5025782 | June 1991 | Salerno |
| 5018514 | May 1991 | Grood et al. |
| 4982732 | January 1991 | Morris |
| 4817588 | April 1989 | Bledsoe |
| 4776326 | October 1988 | Young et al. |
| 4768500 | September 1988 | Mason et al. |
| D291598 | August 1987 | Detty |
| 4655201 | April 1987 | Pirmantgen |
| 4632097 | December 1986 | Brooks |
| 4531515 | July 1985 | Rolfes |
| 4524766 | June 1985 | Petersen |
| 4489718 | December 1984 | Martin |
| 4381768 | May 1983 | Erichsen et al. |
| D265248 | June 1982 | Grigorieff |
| 3805773 | April 1974 | Sichau |
| 2958325 | November 1960 | Clayton |
| 1780959 | November 1930 | Wilkes |
| 1336695 | April 1920 | Gromes |
| 1018452 | February 1912 | Slaughter |
| 649237 | May 1900 | Dyson |
| D265248 | June 1982 | Grigorieff |
| D291598 | August 1987 | Detty |

### Foreign Patent Documents

| | | |
|---|---|---|
| 2414325 | August, 1979 | FR |
| 0 665 160 | August, 1995 | EP |
| 1 086 671 | March, 2001 | EP |
| 19736 | November, 1902 | GB |
| 12997 | September, 1925 | NL |

### BACKGROUND OF THE INVENTION

This invention relates generally to the field of orthopaedic knee braces and more specifically to an apparatus that helps minimize potential knee injuries that stem from typical wear and tear inside the knee joint caused by frequent or prolonged periods of squatting. The present application relates to orthopaedic braces and preventative health care and maintenance adapted with a pneumatic strut system and spring or coil hinge system for use in helping stabilize and cushion the knee joint so as to avoid overuse of the knee or rehabilitate after prior damage to the knee or cartilage.

In order to prevent injury from ever occurring to the human knee joint and to ensure the proper healing of a human joint after an injury or invasive surgery, it is often desirable to limit the pivotal motion of the human knee joint to a predetermined angular range between full extension and full flexion. The pivotal motion may be limited by a range of motion hinge disposed between an upper housing and a lower housing. In order for the orthopedic brace to function properly, the hinge must be adaptable to the body proportions (i.e. weight, height, activity levels) of the wearer. Furthermore, in order to relieve some of the stress and pressure placed on the knee joint, certain devices have been employed to attempt to cushion or absorb such weight and pressure, but nearly all, if not all, of the other devices employ such means after the knee joint is already flexed or contracted. None seem to relieve stress upon the knee on the way down to the crouching position as well as during the crouching position itself.

The following U.S. Patents, which describe orthopedic braces of this general type, are herein incorporated by reference to establish the nature of such braces, and how and why such equipment is used. U.S. Pat. No. 7,097,627, issued on Aug. 29, 2006 to Enzerink et. al. entitled “Orthopedic Brace Having a Range of Motion Hinge with an Adjustable length strut”. U.S. Pat. No. 552,143 issued on Dec. 31, 1895; U.S. Pat. No. 649,237 issued on May 8, 1900; U.S. Pat. No. 4,776,326 issued to Young et al., on Oct. 11, 1988 entitled “Modular Lower Limb Bracing System”; U.S. Pat. No. 4,817,588 issued to Bledsoe on Apr. 4, 1989 entitled “Motion Restraining Knee Brace”; U.S. Pat. No. 4,982,732 issued to Morris on Jan. 8, 1991 entitled “Orthopedic Rehabilitation Knee Brace”; U.S. Pat. No. 5,052,379 issued to Airy et al., on Oct. 1, 1991 entitled “Combination Brace and Wearable Exercise Apparatus for Body Joints”; and U.S. Pat. No. 5,018,514 issued to Grood et al., on May 28, 1991 entitled “Knee Brace”.

The following patent describes a hinge strut assembly mechanism used primarily for the purpose of opening and closing a heavy door. It was issued to Bye, et. al. on Feb. 13, 1990 and is U.S. Pat. No. 4,899,420. No such air or gas strut or suspension system was found for the purpose of cushioning the knee joint in a knee or orthopedic brace.

The orthopedic braces described in the aforementioned incorporated patents are inferior to the present invention. In some cases such knee braces aim to simply rehabilitate an already damaged knee joint. The present application, though, is suitable for rehabilitation of the knee joints of an already injured wearer but also aims to prevent the wear and tear of the knee and surrounding cartilage by cushioning and absorbing the weight and stress away from the knee joint and into the unique combination of the strut and hinge system.

Furthermore, some other devices cannot be adjusted to prevent hyperextension of the knee to suit a particular patient or user, rather, the braces come in various fixed ranges of motion, which might not be suitable for some people. This invention also provides for an adjustable hinge which makes it virtually impossible to hyperextend the knee when adjusted correctly.

It is thus an object of the present invention to provide an orthopaedic brace that can be worn to prolong the usefulness and effectiveness of the knee joint especially where continuous or excessive squatting and crouching occur.

It is thus another object of the present invention to provide an orthopaedic brace that assists the wearer in standing up from the crouching or squatting position as the lift from the pneumatic strut provides such cushion and shock absorption and lift.

It is still yet another object of the present invention to provide an orthopaedic brace that is adjustable to suit the extension and flexion needs of the wearer.

There is a product on the market known as the Kneesaver which attempts to relieve stress to the knee by placing a cushion between the buttock and calf area of the wearer. However, this product can only relieve pressure to the knee when the knees are already fully or almost fully contracted. There is no support or cushion to the knees during the contraction phase of the knees downward nor the flexion phase of the knees upward. It is also cumbersome for any user to sprint or run with the Kneesaver product. A patent for this product was not found among those issued but was found to be on sale on the market.

The present application and invention buffers the effects to the knee before full contraction of the knee joint and almost as soon as the bending of the knees begin. This will both prolong the maximum utility of the knee as well as its longevity.

Most knee brace technology is aimed at rehabilitating an already injured knee. The emphasis should not only be on rehabilitation but on prevention as well. The present invention solves both problems.

AgachaFlex's function and main purpose is to serve and assist the ergonomic need of heavy lower extremity activity by absorbing and redirecting harmful knee stress right back into its own mechanics. AgachaFlex is unique and may soon be identified as the new emerging technology for knee braces.

While typical hinged knee braces manufactured by companies such as DonJoy, Bledsoe, or Innovation Sports all offer support and stability; they're all similar in the sense that they are usually used following an injury or under post-operative necessities (after surgery).

Most hinged braces are made for people who suffer from medial or lateral instability of the knee as well as mild to moderate anterior cruciate or posterior cruciate instabilities as well as meniscus injuries. Not much has been developed by these manufacturers concerning preventative or ergonomic uses for their orthopedic braces. It is usually after the fact of an injury that these devices focus their utility, and even so, the devices are used mostly for stability of the knee and little else. They address gait and flexion extension but seldom do they ever refer to power postures or ergonomics and how these terms correlate with body mechanics. Furthermore, some of these braces can become quite cumbersome to carry around and can only be used for a limited amount of time.

In addition, for someone who frequents this type of power posture, or crouch, such as a professional catcher in baseball, the Agachaflex could mean the preservation or elongation of a lucrative career. Identifying the risk factors and actually taking the necessary steps to help prevent them can mean the difference between enjoying a long, productive career or suffering from an injury that could have been prevented from the outset.

Finally, it is worth noting that meniscus injuries that occur as a result of excessive overuse or extreme wear and tear (friction) between the femur (thigh bone) and tibia (shin bone) and that can potentially cause degeneration in the knee joint, can, in fact, be prevented. Proper body mechanics is crucial in minimizing or eliminating risk factors that can lead to this type of injury. With the application of AgachaFlex and its off-loading and/or weight bearing capabilities, consumers are able to kneel, squat or crouch without the added pressure to their knee joints. Sufferers of osteoarthritis will particularly benefit from the Agachaflex and its breakthrough technology.

### BRIEF SUMMARY OF THE INVENTION

Accordingly, it is a principle object of the present invention to provide a knee brace hinge system combined with a gas or air strut system in order to maximize support and cushion for the knee joint especially during crouching or squatting.

It is another object of the present invention to provide a coil or spring loaded hinge system together with a compressing strut mechanism that includes a swiveling connection to the upper and lower extremity harnesses causing the strut to fold or collapse neatly against the leg as the hinge coil system continues to displace the pressure and stress normally put upon the knee joint.

It is another object of the present invention to provide an adjustable dial or switch on the which will disallow the knee brace and thus the knee joint itself to hyperextend.

It is yet another object of the present invention to provide a air or gas strut system which cushions and absorbs substantially all the shock or impact onto itself and away from the knee joint.

In accordance with the above objects and others which will become apparent hereinafter, there is provided a coil or spring hinge system which mimics the contraction of the knee joint and bends the upper and lower extremity harnesses akin to how the knee joint bends the upper and lower extremities of the legs. The hinge is secured between and divides the upper and lower harness assemblies and is rotatably connected by a hinge pivot means. A gas strut is pivotably mounted to the upper and lower extremity harness and is allowed to freely compress and decompress according to the movement of the wearer. The gas strut is pivotably attached to the upper and lower harnesses respectively. Since it is rotatably connected to a swivel or pivot means, when the wearer crouches or squats, the strut folds neatly aside the leg region as it also approaches near full compression. The hinge system works synchronistically with the strut to offer maximum support, stability and cushion for the knee joint.

The present invention will be better understood and the objects and important features, other than those specifically set forth above, will become apparent when consideration is given to the following details and description, which when taken in conjunction with the annexed drawings, describes, discloses, illustrates, and shows preferred embodiments or modifications of the present invention and what is presently considered and believed to be the best mode of practice in the principles thereof. Other embodiments or modifications may be suggested to those having the benefit to the teachings herein, and such other embodiments of modifications are intended to be reserved especially as they fall within the scope and spirit of the subjoined claims.

Still yet another object of the invention is to provide a knee brace with a simple adjusting hyperextension and flexion control that is adjustable to the specific traits of the individual wearer and simple to adjust.

Another object of the invention is to provide a knee brace that can accommodate a wide range of individual's heights, weights, and activity levels and mobility levels.

Other objects and advantages of the present invention will become apparent from the following descriptions, taken in connection with the accompanying drawings, wherein, by way of illustration and example, an embodiment of the present invention is disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a side perspective view of an adjustable orthopaedic knee brace assembly having a gas or air strut assembly with an adjustable coil or spring-loaded hinge member that embodies principles of the present invention showing the brace operatively connected to a human leg;

FIG. 2 is a Plain side view of the invention showing a subject wearing the knee or orthopedic brace with the knee joint contracted and the body crouching.

FIG. 3 is a cross-sectional view of the orthopedic strut and hinge assembly mechanism.

### Corresponding reference characters indicate corresponding parts throughout the several views.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

Reference is now made specifically to the drawings in which identical or similar parts are designated by the same reference numerals throughout.

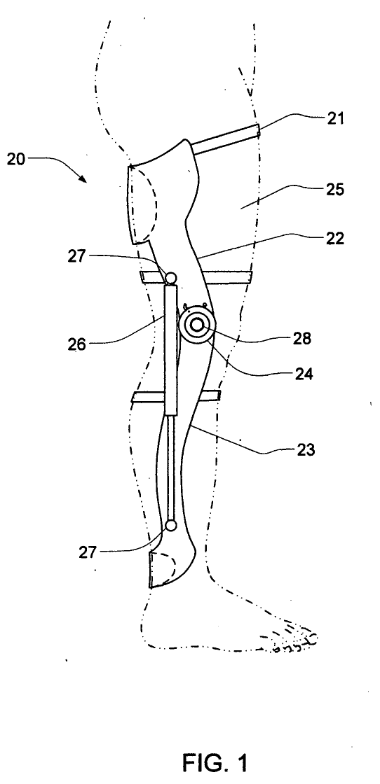

Referring to FIG. 1, an orthopedic brace 20 is shown operatively attached to a human leg 25 using a plurality of straps 21 mounted on an upper extremity harness 22 and a lower extremity harness 23 with a coil or spring-loaded hinge assembly system 24 disposed between the upper extremity harness 21 and the lower extremity harness 23. The orthopaedic brace 20 (i.e. the hinge assembly 24, the upper extremity harness 22, and the lower extremity harness 23 or “assembly”) is deliberately shown from this perspective along the outside of the human leg 25 and there is no mirror image of the device necessary for the inside part of the leg. Dotted lines at the top portion of the upper harness 23 and bottom portion of the lower harness 23 depicts how the brace adheres to the opposite or inside of the leg, secured by the plurality of straps 21. (However, of course, there is an identical complete brace assembly 20 necessary for the other human leg).

A single strut 26 is pivotably mounted for swiveling capability and attached to the upper and lower extremity harnesses respectively 27 so that upon bending of the knee, the strut folds or collapses neatly aside the leg.

A dial or knob 28 is shown which is used to adjust the brace 20 in order to avoid hyperextension of the knee joint. That is, for instance, the brace can be set to allow only a slight degree of extension of the knee joint.

During the contraction of the knee, the strut 26 is allowed to telescope, or move in and out of its housing chamber and provides the cushion and shock absorption which is the object of the present invention.

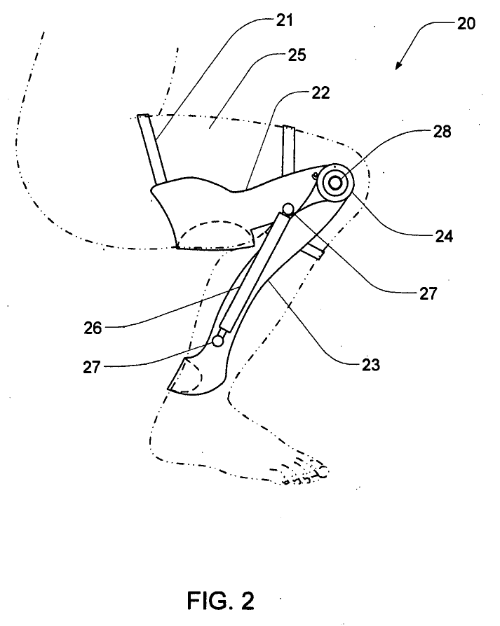

Referring to FIG. 2, the human leg 25 is shown bent with the subject wearer's body represented in a crouching or squatting position.

The strut 26 is shown near full compression wherein it lays substantially parallel to the subject user's side of the leg. The attachments to the upper and lower harnesses 27 have swiveled and allowed the strut to pivot in such a manner.

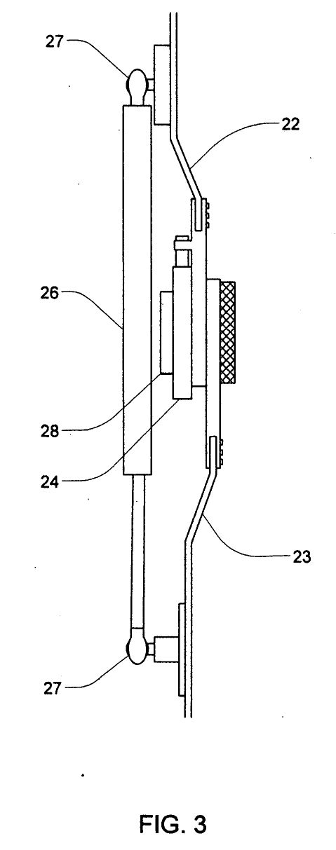

Referring to FIG. 3, a cross-sectional front view of the brace 20 is shown with an adjustable knob or dial 28 on the hinge system 24.

Although the invention has been described in detail with reference to a preferred embodiment and an alternative embodiment, variations and modifications exist within the scope and spirit of the invention. Additional features of the invention will become apparent to those skilled in the art upon consideration of the detailed description of preferred embodiments exemplifying the best mode of carrying out the invention as presently perceived.

What is claimed is:

1. An orthopedic brace, comprising a strut device pivotably attached to an upper and lower extremity harness divided by a pivotably mounted hinge mechanism, comprising:
a) A first upper extremity harness support housing;
b) A second lower extremity harness support housing;
c) An adjustable hinge mechanism pivotably connecting the first and second harnesses,
d) A strut device that is pivotably connected to the upper and lower extremity harnesses respectively.

2. The orthopedic brace of claim 1 wherein said strut is a gas or air powered strut that is connected to the brace which contracts and expands to provide cushioning to the knees.

3. The orthopedic brace of claim 1 wherein said hinge mechanism is a coil or spring-loaded device which provides further resistance and shock absorption away from the knee joint.

4. The orthopedic brace of claim 1 wherein said hinge provides an adjustable means whereby the user or wearer is able to adjust the mechanism to prevent hyperextension of the knee joint and said adjustable hinge system works in conjunction with the strut for maximum mobility and cushioning;

5. An orthopedic knee brace comprising: an adjustable coil-loaded spring hinge connected to an upper and lower section housing which prevents hyperextension of the knee and provides maximum support while squatting or crouching; and an air, gas or other gaseous element strut including being connected with a rotatable pivoting mechanism to an upper and lower housing harness assembly respectively.

6. The combination of a hinge mechanism and strut system according to claim 1, wherein at least one strut is a pneumatic strut including a pneumatic cylinder which moves in a piston-like fashion in and out of a larger circumferenced housing and said pneumatic cylinder is longitudinally-connected where one cylindrical portion telescopes, or moves in and out of, another cylindrical member and is slidably mounted to move within an aperture of a larger strut housing.

7. The orthopedic knee brace of claim 5, wherein the air or gas powered strut is attached to the main housing and collapses into its sheath as the knee bends or contracts and after the strut compresses substantially and folds substantially close to the leg a discus coil or spring loaded hinging mechanism takes over and provides the support and shock absorption to relieve the stress and pressure off the knee joint.

8. The orthopedic knee brace of claim 5, wherein the progression from contraction to extension of the knee is aided simultaneously by the recoil from the hinge system and the lift from the said strut thus further relieving the knee of having to push the body's weight upward; the strut then continues to slide out of its sheath until the knee is fully extended until the point whereby the adjustable hinge system has been set so as to prevent the hyperextension of the knee joint.

9. The orthopedic knee brace of claim 5, wherein the strut system pivots rotatably about an axis provided by its connection to the main housing of molded plastic or composite which is shaped around the upper and lower extremity of the leg appendage and forthwith collapses or folds substantially along the leg after substantially full contraction of the knee and near full compression of the strut mechanism.

10. The orthopedic knee brace of claim 5, wherein the hinging assembly bends simultaneously with the collapsing of the strut mechanism and after the strut mechanism is fully compressed and folded substantially alongside subject wearer's leg, said hinging device with its coil or spring loaded action resists the pressure and stress of the weight of the body and gravity normally put on the knee joints and absorbs that pressure, stress and strain until when the leg is extended and the wearer stands upright, the energy absorbed by the hinge system is displaced to aid in lifting the body's weight against gravity further dampening the effects of the body's weight and gravity usually put upon the knee joint.

11. The orthopedic knee brace of claim 1, wherein the strut is slidable within a longitudinal channelled sheath opening fixably and rotatably attached to the main housing, and, when the subject wearer is standing upright, the strut is substantially perpendicular to the ground.

12. The orthopedic knee brace of claim 1, wherein the strut pivots, folds and collapses neatly to a position alongside the leg utilizing the pivoting or swiveling action of the rotatably connected attachments to the upper and lower extremity housing harnesses.
